(19) United States
(12) Patent Application Publication
(10) Pub. No.: US 2025/0005752 A1
(43) Pub. Date: Jan. 2, 2025

(54) MEDICAL DATA PROCESSING APPARATUS AND METHOD

(71) Applicant: CANON MEDICAL SYSTEMS CORPORATION, Tochigi (JP)

(72) Inventors: Shuki MARUYAMA, Nasushiobara (JP); Hidenori TAKESHIMA, Taito-ku (JP)

(73) Assignee: CANON MEDICAL SYSTEMS CORPORATION, Tochigi (JP)

(21) Appl. No.: 18/754,281

(22) Filed: Jun. 26, 2024

(30) Foreign Application Priority Data
Jun. 30, 2023 (JP) 2023-108307

(51) Int. Cl. G06T 7/00; G16H 50/70

(52) U.S. Cl. CPC G06T 7/0012; G16H 50/70; G06T 2207/10088; G06T 2207/30096

(57) ABSTRACT

A medical data processing apparatus according to an embodiment includes processing circuitry. The processing circuitry is configured to receive medical data as an input. The processing circuitry is configured to perform a medical determination process on the basis of the input data. The processing circuitry is configured to output information about a determination step together with a determination result of the medical determination process.

### CROSS-REFERENCE TO RELATED APPLICATIONS

This application is based upon and claims the benefit of priority from Japanese Patent Application No. 2023-108307, filed on Jun. 30, 2023; the entire contents of which are incorporated herein by reference.

### FIELD

Embodiments described herein relate generally to a medical data processing apparatus and a method.

### BACKGROUND

A technique for providing medical classification by using a Magnetic Resonance (MR) image and an MR spectrum is known. According to the technique, an output is a “grade”, while the classification (a determination process) is carried out in a black box. For this reason, a problem is recognized where users such as medical doctors who determine a disease are unable to make an interpretation according to a guideline.

### BRIEF DESCRIPTION OF THE DRAWINGS

FIG. 1 is a diagram illustrating an exemplary configuration of a medical data processing system and a medical data processing apparatus according to a first embodiment;

FIG. 2 is a chart for explaining medical classification carried out by a determining function of the medical data processing apparatus according to the first embodiment;

FIG. 3 is a drawing for explaining details of an example of a trained model according to the first embodiment;

FIG. 4 is a drawing for explaining an example of processes performed by a visualizing function according to the first embodiment;

FIG. 5 is a flowchart illustrating an example of a flow in processes performed by the medical data processing apparatus according to the first embodiment;

FIG. 6 is a drawing for explaining an example of processes performed by a medical data processing apparatus according to a modification example of the first embodiment;

FIG. 7 is a diagram illustrating an exemplary configuration of a medical data processing system and a medical data processing apparatus according to a second embodiment;

FIG. 8A is a drawing illustrating an example of display content displayed on a display according to the second embodiment;

FIG. 8B is a drawing illustrating another example of the display content displayed on the display according to the second embodiment;

FIG. 8C is a drawing illustrating yet another example of the display content displayed on the display according to the second embodiment;

FIG. 8D is a drawing illustrating yet another example of the display content displayed on the display according to the second embodiment;

FIG. 9A is a drawing illustrating yet another example of the display content displayed on the display according to the second embodiment;

FIG. 9B is a drawing illustrating yet another example of the display content displayed on the display according to the second embodiment;

FIG. 9C is a drawing illustrating yet another example of the display content displayed on the display according to the second embodiment; and

FIG. 9D is a drawing illustrating yet another example of the display content displayed on the display according to the second embodiment.

### DETAILED DESCRIPTION

One of the problems to be solved by the embodiments set forth in the present disclosure is to present a user who determines a disease with information that assists the determination process in a form that is interpretable by the user. However, the problems to be solved by the embodiments set forth in the present disclosure are not limited to the problem described above. It is also possible to consider problems corresponding to advantageous effects achieved by the configurations described in the following embodiments as other problems.

A medical data processing apparatus according to an embodiment includes processing circuitry. The processing circuitry is configured to receive medical data as an input. The processing circuitry is configured to perform a medical determination process on the basis of the input data. The processing circuitry is configured to output information about a determination step together with a determination result of the medical determination process.

Exemplary embodiments and modification examples of a medical data processing apparatus and a method will be explained in detail below, with reference to the accompanying drawings. The medical data processing apparatus and the method of the present disclosure are not limited by the embodiments and the modification examples described below. Further, it is acceptable to combine each of the embodiments with any of the other embodiments, the modification examples, and conventional techniques, as long as no conflict occurs in the processing. Similarly, it is also acceptable to combine each of the modification examples with any of the embodiments, the other modification examples, and conventional techniques, as long as no conflict occurs in the processing.

### First Embodiment

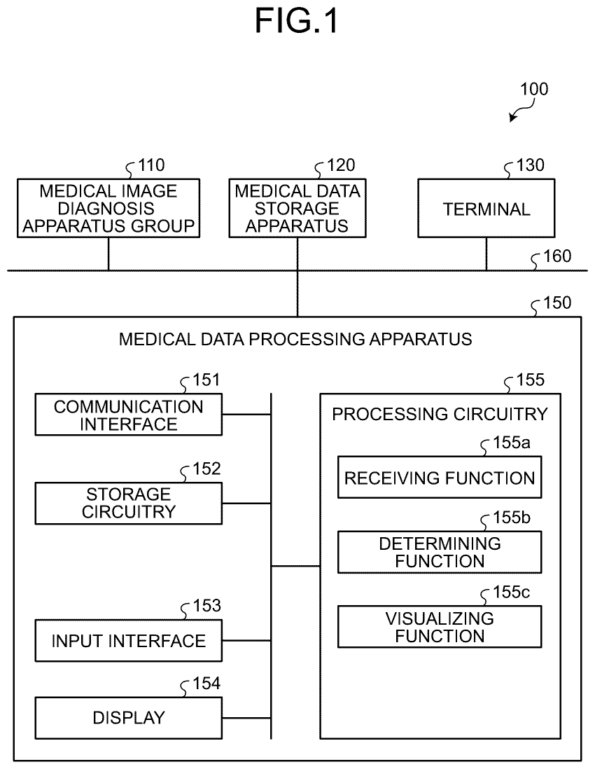

FIG. 1 is a diagram illustrating an exemplary configuration of a medical data processing system 100 and a medical data processing apparatus 150 according to a first embodiment.

As illustrated in FIG. 1, the medical data processing system 100 according to the present embodiment includes a medical image diagnosis apparatus group 110, a medical data storage apparatus 120, a terminal 130, and a medical data processing apparatus 150. In this situation, the apparatuses are connected so as to be able to communicated with one another via a network 160. The medical data processing system 100 and the medical data processing apparatus 150 according to the present embodiment are installed in a medical facility such as a hospital or a clinic, for example, and are configured to assist diagnosing processes performed by a user such as a medical doctor.

The medical image diagnosis apparatus group 110 is a set made up of a plurality of medical image diagnosis apparatuses. Examples of the medical image diagnosis apparatuses include various types of medical image diagnosis apparatuses such as a Magnetic Resonance Imaging (MRI) apparatus, an X-ray Computed Tomography (CT) diagnosis apparatus, an ultrasound diagnosis apparatus, an X-ray diagnosis apparatus, a Positron Emission Tomography (PET) apparatus, and a Single Photon Emission Computed Tomography (SPECT) apparatus.

The MRI apparatus is configured to generate a Magnetic Resonance (MR) image and an MR spectrum. The X-ray CT apparatus is configured to generate a CT image. The ultrasound diagnosis apparatus is configured to generate an ultrasound image. The X-ray diagnosis apparatus is configured to generate an X-ray image and an angiography image. The PET apparatus is configured to generate a PET image. The SPECT apparatus is configured to generate a SPECT image. The MR image, the MR spectrum, the CT image, the ultrasound image, the X-ray image, the angiography image, the PET image, and the SPECT image are each medical data and are each data acquired by the medical image diagnosis apparatus. Further, the MR image, the CT image, the ultrasound image, the X-ray image, the angiography image, the PET image, and the SPECT image are each also a medical image.

The medical data storage apparatus 120 is configured to store therein various types of medical data. More specifically, the medical data storage apparatus 120 is configured to obtain the medical data from the medical image diagnosis apparatus group 110 via the network 160 and to store the obtained medical data by saving the data into storage circuitry within the medical data storage apparatus 120. Further, for example, the medical data storage apparatus 120 is realized by using a Picture Archiving and Communication System (PACS) or the like and is configured to store the medical data in a format compliant with a Digital Imaging and Communications in Medicine (DICOM) scheme. The medical data storage apparatus 120 is realized by using a computer machine such as a server or a workstation.

The terminal 130 is a terminal used by the user and is configured to display various types of medical data. For example, the terminal 130 is configured to obtain the various types of medical data such as the medical images, MR spectra, and results of medical classification, from the medical data storage apparatus 120 and the medical data processing apparatus 150 via the network 160. Further, the terminal 130 is configured to cause a display included in the terminal 130 to display the obtained medical data. The display included in the terminal 130 is an example of a display unit. For example, the terminal 130 is realized by using a computer machine such as a workstation, a personal computer, or a tablet terminal.

The medical data processing apparatus 150 is configured to perform a medical determination process including various types of medical classification, on the basis of the medical data. In other words, the medical data processing apparatus 150 is configured to carry out the medical classification and to perform the medical determination process on the basis of the medical data. In the following description, the actions of the medical data processing apparatus 150 carrying out the medical classification and the medical determination process may collectively be referred to as “performing the medical determination process”. For example, the medical data processing apparatus 150 is configured to obtain the medical data from either the medical image diagnosis apparatus group 110 or the medical data storage apparatus 120 via the network 160. Further, on the basis of the obtained medical data, the medical data processing apparatus 150 is configured to perform various types of medical determination processes. For example, the medical data processing apparatus 150 is realized by using a computer machine such as a server or a workstation. Further, in the following sections, an example will be explained in which the single medical data processing apparatus (i.e., the medical data processing apparatus 150) performs the various types of processes described below; however, it is also acceptable to configure a plurality of medical data processing apparatuses 150 to perform the various types of processes described below in a distributed manner.

As illustrated in FIG. 1, the medical data processing apparatus 150 includes a communication interface 151, storage circuitry 152, an input interface 153, a display 154, and processing circuitry 155.

The communication interface 151 is configured to control transfer and communication of various types of information and various types of data transmitted and received between the medical data processing apparatus 150 and other apparatuses connected to the medical data processing apparatus 150 via the network 160. The communication interface 151 is connected to the processing circuitry 155. The communication interface 151 is configured to receive data transmitted by the other apparatuses. In that situation, the communication interface 151 is configured to transmit the received data to the processing circuitry 155. Also, the communication interface 151 is configured to receive data transmitted by the processing circuitry 155. In that situation, the communication interface 151 is configured to transmit the received data to any of the other apparatuses. For example, the communication interface 151 is realized by using a network card, a network adaptor, a Network Interface Controller (NIC), or the like.

The storage circuitry 152 is configured to store therein various types of data and various types of programs. The storage circuitry 152 is connected to the processing circuitry 155. Under control of the processing circuitry 155, the storage circuitry 152 is configured to store therein data transmitted thereto by the processing circuitry 155. Further, any of the data stored in the storage circuitry 152 can be read by the processing circuitry 155. For example, the storage circuitry 152 is realized by using a semiconductor memory element such as a Random Access Memory (RAM) or a flash memory, or a hard disk, an optical disk, or the like.

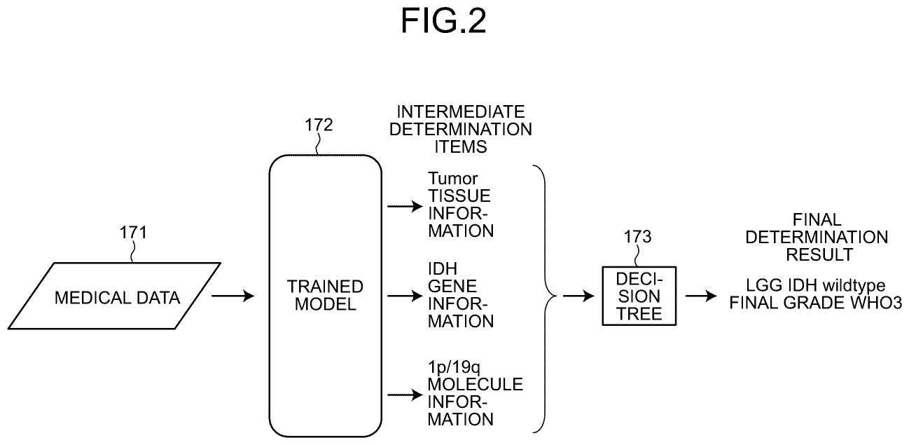

The storage circuitry 152 has stored therein a trained model 172 (explained later; see FIG. 2) and a decision tree 173 (explained later; see FIG. 2). The storage circuitry 152 is an example of a storage unit, for instance.

The input interface 153 is configured to receive operations to input various types of instructions and various types of information from the user such as a medical doctor. The input interface 153 is connected to the processing circuitry 155. The input interface 153 is configured to convert the operations received from the user into electrical signals and to transmit the electrical signals to the processing circuitry 155. For example, the input interface 153 is realized by using a trackball, a switch button, a mouse, a keyboard, a touchpad configured to receive operations as being touched on an operation surface thereof, a touch screen in which a display screen and a touchpad are integrally formed, a contactless input interface using an optical sensor, an audio input interface, and/or the like. In the present disclosure the input interface 153 does not necessarily need to include physical operation component parts such as the mouse, the keyboard, and/or the like. For instance, possible examples of the input interface 153 include electrical signal processing circuitry configured to receive an electrical signal corresponding to an input operation from an external input mechanism provided separately from the apparatus and to transmit the electrical signal to the processing circuitry 155.

The display 154 is configured to display various types of information and various types of data. The display 154 is connected to the processing circuitry 155. Under control of the processing circuitry 155, the display 154 is configured to display various types of information and various types of data transmitted thereto by the processing circuitry 155. For example, the display 154 is realized by using a liquid crystal display, a Cathode Ray Tube (CRT) display, a touch panel, or the like. The display 154 is an example of a display unit.

The processing circuitry 155 is configured to control the entirety of the medical data processing apparatus 150. For example, the processing circuitry 155 is configured to carry out the various types of medical classification in accordance with the operations received from the user via the input interface 153. Further, for example, the processing circuitry 155 is configured to obtain the medical data transmitted thereto by either the medical image diagnosis apparatus group 110 or the medical data storage apparatus 120 via the network 160. Further, the processing circuitry 155 is configured to store the obtained medical data into the storage circuitry 152.

As illustrated in FIG. 1, the processing circuitry 155 includes a receiving function 155a, a determining function 155b, and a visualizing function 155c.

For example, the processing circuitry 155 is realized by using a processor. In that situation, the abovementioned processing functions are stored in the storage circuitry 152 in the form of computer-executable programs (medical data processing programs). Further, the processing circuitry 155 is configured to realize the processing functions corresponding to the programs, by reading the programs stored in the storage circuitry 152 and executing the read programs. In other words, the processing circuitry 155 that has read the programs has the processing functions illustrated in FIG. 1.

Alternatively, the processing circuitry 155 may be structured by combining together a plurality of independent processors, so that the processing functions are realized as a result of the processors executing the programs. Further, the processing functions of the processing circuitry 155 may be realized as being distributed among or integrated into one or more pieces of processing circuitry. Further, the processing functions of the processing circuitry 155 may be realized by a combination of hardware such as circuitry and software. Furthermore, although the example was explained above in which the programs corresponding to the processing functions are stored in the single piece of circuitry (i.e., the storage circuitry 152), the programs may be stored in a plurality of pieces of storage circuitry in a distributed manner. For example, it is acceptable to store the programs corresponding to the processing functions in the plurality of pieces of storage circuitry in a distributed manner, so that the processing circuitry 155 reads and executes the programs from the pieces of storage circuitry.

The exemplary configuration of the medical data processing system 100 and the medical data processing apparatus 150 according to the present embodiment has thus been explained. As explained below, the medical data processing system 100 and the medical data processing apparatus 150 according to the present embodiment are configured to present the user such as a medical doctor who determines a disease with information that assists the determination process, in a form that is interpretable by the user.

The receiving function 155a is configured to obtain the medical data from either the medical image diagnosis apparatus group 110 or the medical data storage apparatus 120 via the network 160. Also, the receiving function 155a is configured to receive the medical data as an input to the trained model 172. The receiving function 155a is an example of a receiving unit, for instance.

FIG. 2 is a chart for explaining medical classification carried out by the determining function 155b of the medical data processing apparatus 150 according to the first embodiment. As illustrated in FIG. 2, the determining function 155b is configured to perform a medical determination process, by applying, to medical data 171, a medical determination model (a medical classification model) including the trained model 172 and the decision tree 173. In other words, the determining function 155b is configured to perform the medical determination process on the basis of the medical data 171. The medical data 171 includes data of a tissue of an examined subject (hereinafter, “patient”) to be processed.

The determining function 155b according to the present embodiment is configured to perform the medical determination process on the basis of one of more pieces of medical data 171. More specifically, the determining function 155b is configured to output a medical determination result (a medical classification result) related to the patient, by applying the one or more pieces of medical data 171 to the medical determination model including the trained model 172 and the decision tree 173. The medical determination result is information indicating a result of the medical determination process related to the patient of the medical data. As the medical determination result, for example, a grade of a disease affecting the patient may be output. For example, the determining function 155b is an example of a determining unit.

The medical data 171 applied to the medical determination model including the trained model 172 and the decision tree 173 may be any of various types of data. For example, the medical data 171 may be data obtained by any of the various types of medical image diagnosis apparatuses listed above. Further, the medical data 171 may be a result of any of various types of tests such as a gene test, a blood test, a pathological test, and a molecular test. Further, the medical data 171 may be a result of an examination performed by any of various types of image diagnosis apparatuses.

Furthermore, the medical data 171 may be information (morphological information) related to morphology of a site of the patient to be processed, may be information (composition information) related to a composition of the site of the patient to be processed, or may be information (property information) related to a physical property of the site of the patient to be processed. Alternatively, the medical data 171 may be physiological information of the site of the patient to be processed.

In the following sections, an example will be explained in which, as the medical determination process, the medical data processing apparatus 150 is configured to determine a grade of a glioma, by using the decision tree 173 based on a brain tumor classification according to the World Health Organization (WHO), which is a medical guideline for classifying grades of gliomas that are brain tumors. However, possible examples of the medical determination process performed by the medical data processing apparatus 150 are not limited to this example.

As illustrated in FIG. 2, the trained model 172 is a machine learning model of which parameters have been trained so as to receive an input of the one or more pieces of medical data 171 and to output one or more intermediate determination items. The medical data 171 includes information about a glioma cell of the brain, which is the patient's tissue to be processed. When two or more pieces of medical data 171 are input to the trained model 172, the two or more pieces of medical data 171 including information about the glioma cell of the brain of the same patient are input to the trained model 172. As the trained model or the machine learning model according to the first embodiment, it is possible to use a classifier or a discriminator such as a neural network, a deep neural network, a support vector machine, a random forest scheme, or the like. As illustrated in FIG. 2, the intermediate determination items are, more specifically, tissue information, gene information, and molecule information related to the patient.

The tissue information is information including: information (tumor presence information) indicating whether or not a glioma cell of the brain represented by the patient's tissue to be processed has a tumor (a brain tumor, a glioma); and information (grade information) provisionally indicating a grade of the tumor. For example, when it is estimated that the glioma cell has a tumor, the tumor presence information indicates “1”. On the contrary, when it is estimated that the glioma cell does not have a tumor, the tumor presence information indicates “0”. For example, the trained model 172 is configured to calculate a probability that the glioma cell may have a tumor. The probability is expressed with a value within the range from 0 to 1 inclusive. The higher the probability that the glioma cell may have a tumor is, the closer is the probability value to “1”. The lower the probability that the glioma cell may have a tumor is, the closer is the probability value to “0”. The probability is reliability indicating a likelihood of having a tumor. Further, the trained model 172 is configured to compare the probability with a prescribed threshold value (a determination reference value) and to generate the tumor presence information indicating “1”, when the probability is equal to or higher than the determination reference value. On the contrary, the trained model 172 is configured to generate the tumor presence information indicating “0”, when the probability is lower than the determination reference value.

Further, when the tumor presence information indicates “1” (i.e., when a tumor is present), the trained model 172 is configured to roughly estimate a grade of the tumor and to generate the grade information indicating the estimated grade.

After that, when having generated the tumor presence information and the grade information, the trained model 172 is configured to output the tissue information including the tumor presence information and the grade information.

The gene information is information indicating whether or not a gene that codes isocitrate dehydrogenase (IDH) in the glioma cell has a mutation. For example, when it is estimated that the gene that codes IDH in the glioma cell has a mutation, the gene information indicates “1”. On the contrary, when it is estimated that the gene that codes IDH in the glioma cell does not have a mutation, the gene information indicates “0”. For example, the trained model 172 is configured to calculate a probability that the gene that codes IDH in the glioma cell may have a mutation. The probability is expressed with a value within the range from 0 to 1 inclusive. The higher the probability that the gene that codes IDH in the glioma cell may have a mutation is, the closer is the probability value to “1”. The lower the probability that the gene that codes IDH in the glioma cell may have a mutation is, the closer is the probability value to “0”. The probability is reliability indicating a likelihood of a mutation occurring in the gene that codes IDH in the glioma cell. Further, the trained model 172 is configured to compare the probability with a determination reference value and to output the gene information indicating “1”, when the probability is equal to or higher than the determination reference value. On the contrary, the trained model 172 is configured to output the gene information indicating “0”, when the probability is lower than the determination reference value.

The molecule information is information indicating whether or not the short arm of chromosome 1 (1p) and the long arm of chromosome 19 (19q) are both deleted from the glioma cell. For example, when it is estimated that the short arm of chromosome 1 and the long arm of chromosome 19 are both deleted from the glioma cell, the molecule information indicates “1”. On the contrary, when it is estimated that at least one of the short arm of chromosome 1 and the long arm of chromosome 19 is not deleted from the glioma cell, the molecule information indicates “0”. For example, the trained model 172 is configured to calculate a probability that the short arm of chromosome 1 and the long arm of chromosome 19 are both deleted from the glioma cell. The probability is expressed with a value within the range from 0 to 1 inclusive. The higher the probability that the short arm of chromosome 1 and the long arm of chromosome 19 are both deleted from the glioma cell is, the closer is the probability value to “1”. The lower the probability that the short arm of chromosome 1 and the long arm of chromosome 19 are both deleted from the glioma cell is, the closer is the probability value to “0”. The probability is reliability indicating a likelihood of the deletion occurring on both the short arm of chromosome 1 and the long arm of chromosome 19. Further, the trained model 172 is configured to compare the probability with a determination reference value and to output the molecule information indicating “1”, when the probability is equal to or higher than the determination reference value. On the contrary, the trained model 172 is configured to output the molecule information indicating “0”, when the probability is lower than the determination reference value.

An example of a method for generating the trained model 172 will be explained. The trained model 172 is a trained machine learning model obtained by causing a machine learning model to perform a machine learning process according to a model training program, on the basis of training data in which medical data including information about a glioma cell of a patient is used as input data, whereas information indicating whether or not the glioma cell has a tumor, information indicating, if the glioma cell has a tumor, a grade of the tumor, information indicating whether or not a gene that codes IDH in the glioma cell has a mutation, and information indicating whether or not the short arm of chromosome 1 and the long arm of chromosome 19 are both deleted from the glioma cell are used as correct answer data. The trained model 172 is generated by a training apparatus. For example, the training apparatus is connected to the medical data processing apparatus 150 via the network 160. Further, the trained model 172 generated by the training apparatus is provided for the medical data processing apparatus 150 via the network 160.

The training apparatus includes the machine learning model such as a Convolution Neural network (CNN). By performing a training process (a supervised training process) based on the training data related to glioma cells of a single patient, the training apparatus generates the trained model 172. At the time of an inference process, the trained model 172 is functioned, upon receipt of an input of medical data corresponding to the input data, so as to output tissue information, gene information, and molecule information (output data) corresponding to the correct answer data. Alternatively, the medical data processing apparatus 150 may have functions similar to those of the training apparatus so that, instead of the training apparatus, the medical data processing apparatus 150 is configured to generate the trained model 172.

An example will be explained in which the machine learning model is a CNN. In that situation, in the training apparatus, the input data is input to the CNN serving as the machine learning model. By applying the CNN to the input data, the training apparatus generates the output data. Thus, the output data is output from the CNN. In the training apparatus, the output data enters an evaluating function. Further, in the training apparatus, the abovementioned correct answer data is also input to the evaluating function. The training apparatus evaluates the output data generated by the CNN on the basis of the input data and the correct answer data, by employing the evaluating function. For example, the evaluating function is configured to compare the generated output data with the correct answer data and to correct coefficients (network parameters such as a weight and a bias, etc.) of the CNN by implementing an error backpropagation method. The evaluation by the evaluating function is given to the CNN as feedback. The training apparatus is configured to repeatedly perform a series of supervised training based on the training data as described above, until the error between the output data and the correct answer data becomes equal to or smaller than a prescribed threshold value, for example. The training apparatus is capable of outputting the trained machine learning model as the trained model 172.

The decision tree 173 is a machine learning model trained so as to receive an input of tissue information, gene information, and molecule information and to output a grade of a glioma. For example, the decision tree 173 is a trained machine learning model obtained by causing a machine learning model to perform a machine learning process according to a model training program, while using a set of tissue information, gene information, and molecule information and a grade of a glioma obtained as a result of carrying out, on the set of information, the brain tumor classification of the World Health Organization serving as a medical guideline for determining (classifying) the grade of the glioma, which is a brain tumor. In other words, the abovementioned medical guideline is used at the time of generating the decision tree 173. As illustrated in FIG. 2, upon receipt of an input of the tissue information, the gene information, and the molecule information output from the trained model 172, the decision tree 173 is configured to output a grade of the glioma, as a final determination result.

In the decision tree 173, various types of conditions are defined with respect to various types of information such as the tissue information, the gene information, and the molecule information. The various types of conditions are defined as either “Yes” or “No”. For this reason, the decision tree 173 is a model (an interpretable model) configured to make it possible to interpret determination steps (classification steps, inference steps) for the user such as a medical doctor who determines a disease. Alternatively, in place of the decision tree 173, it is also acceptable to use a model having functions similar to those of the decision tree 173. For example, it is acceptable to use a random forest scheme, a Naive Bayes tree, a decision list, a decision graph, a Bayesian network, and/or a gradient boosting tree. Similarly to the decision tree 173, the random forest scheme, the Naive Bayes tree, the decision list, the decision graph, the Bayesian network, and the gradient boosting tree are each a model configured to make it possible to interpret the determination steps (the classification steps) for the user such as a medical doctor who determines a disease.

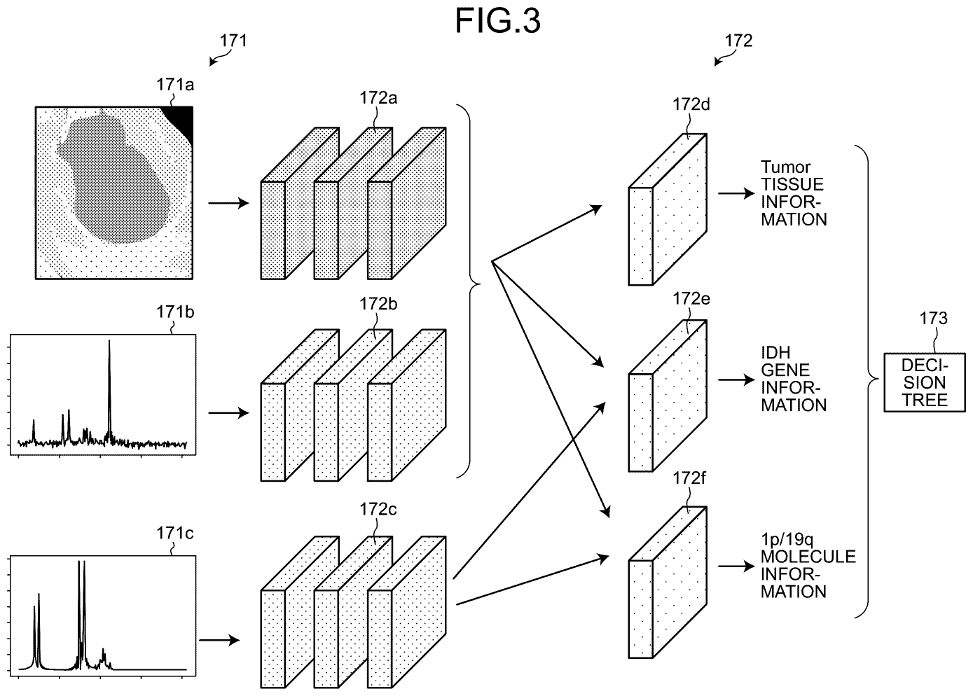

FIG. 3 is a drawing for explaining details of an example of the trained model 172 according to the first embodiment. The trained model 172 illustrated in FIG. 3 is configured to receive an input of three pieces of medical data 171a, 171b, and 171c and to output tissue information, gene information, and molecule information. In the example in FIG. 3, the three pieces of medical data 171a, 171b, and 171c are each MR data. As explained in detail later, the medical data 171a is an MR image, while the medical data 171b and 171c are each an MR spectrum. Accordingly, in the following sections, the medical data 171a may be referred to as an MR image 171a, while the medical data 171b and 171c may be referred to as MR spectra 171b and 171c.

The trained model 172 illustrated in FIG. 3 may be a neural network, for example, and includes an input layer, an intermediate layer, and an output layer. However, illustration of the input layer and the output layer is omitted from FIG. 3. In the example in FIG. 3, the intermediate layer includes a CNN layer, a fully-connected layer, and a pooling layer (not illustrated), or the like.

In the example in FIG. 3, the CNN layer includes three nodes 172a, 172b, and 172c. To the node 172a, the MR image 171a is input via the input layer as medical data 171. The MR image 171a is a morphological image indicating morphology of a glioma cell of the patient. For example, the MR image 171a may be a T1-weighted image, a T2-weighted image, or a Fluid Attenuated Inversion Recovery (FLAIR) image. When the MR image 171a is a two-dimensional image, the node 172a being two-dimensional may be used. In contrast, when the MR image 171a is a three-dimensional image, the node 172a being three-dimensional may be used. The node 172a is configured to receive the input of the MR image 171a and to output a feature value a.

To the node 172b, the MR spectrum 171b is input via the input layer as medical data 171. Further, to the node 172c, the MR spectrum 171c is input via the input layer as medical data 171. In this situation, the MR spectrum 171b is an MR spectrum acquired by an MRI apparatus while implementing a PRESS method. In contrast, the MR spectrum 171c is an MR spectrum acquired by an MRI apparatus while implementing a MEGA-PRESS method. Because the MR spectrum 171b and the MR spectrum 171c are each a one-dimensional spectrum, the node 172b and the node 172c each being one-dimensional may be used. The node 172b is configured to receive the input of the MR spectrum 171b and to output a feature value b. The node 172c is configured to receive the input of the MR spectrum 171c and to output a feature value c.

In the example in FIG. 3, the fully-connected layer includes three nodes 172d, 172e, and 172f. The node 172d is configured to receive an input of the feature value a and the feature value b and to output tissue information via the output layer. The node 172e is configured to receive an input of the feature value a, the feature value b, and the feature value c and to output gene information via the output layer. The node 172f is configured to receive an input of the feature value a, the feature value b, and the feature value c and to output molecule information via the output layer.

In the example in FIG. 3, the example was explained in which the MR image 171a being the morphological image indicating morphology of the glioma cell of the patient is used as the medical data 171 input to the node 172a. It is, however, also acceptable to use other images such as a CT image, an ultrasound image, or an X-ray image being a morphological image indicating morphology of the glioma cell of the patient, as the medical data 171 input to the node 172a.

In addition, the medical data 171 applied to the trained model 172 does not necessarily need to be data obtained by a medical image diagnosis apparatus or the like and may be artificial data generated artificially. For example, as the artificial data, medical data obtained through a simulation, deep learning, or the like may be applied to the trained model 172. More specifically, MR data of an MR image or an MR spectrum may be generated through a physical computation called a Bloch simulator or an MR simulator, so that the generated MR data is applied to the trained model 172. As another example, data indicating behaviors of molecules obtained through a molecular dynamics simulation may be applied to the trained model 172. As yet another example, values converted through a physical computation from actual measurement values obtained by a medical image diagnosis apparatus or the like may be applied to the trained model 172.

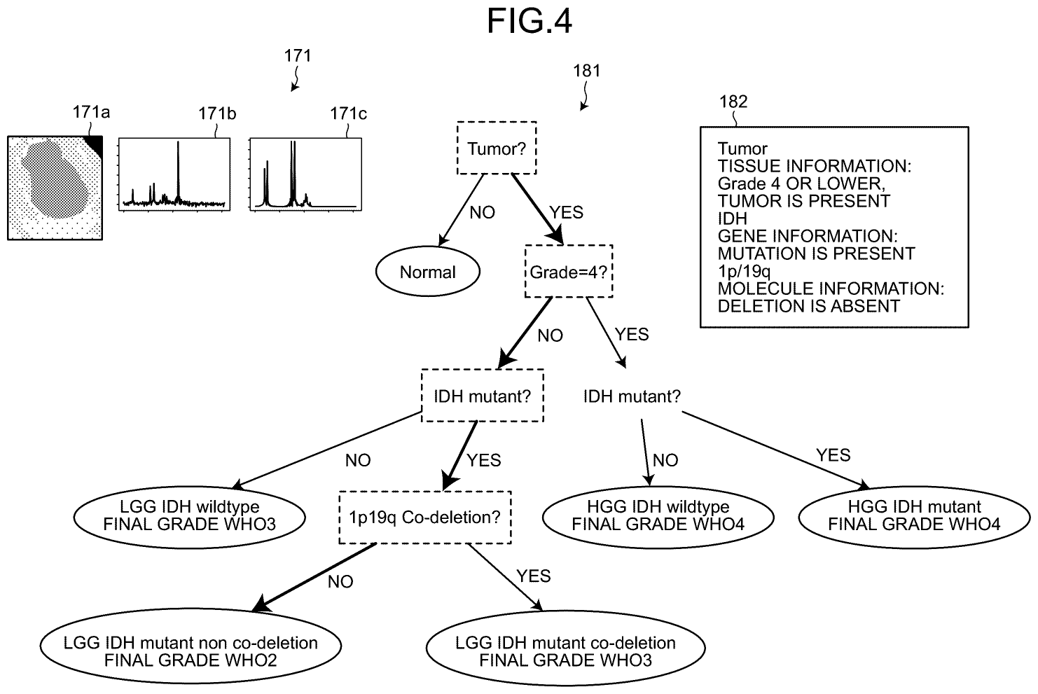

The visualizing function 155c is configured to visualize the determination steps (the classification steps) of the medical determination process (a medical classifying process) performed by the determining function 155b. FIG. 4 is a drawing for explaining an example of processes performed by the visualizing function 155c according to the first embodiment. FIG. 4 illustrates an example of display content displayed on the display 154 by the visualizing function 155c.

For example, as illustrated in FIG. 4, the visualizing function 155c is configured to cause the display 154 to display details 181 of the decision tree 173. Among the details 181 of the decision tree 173, the branching conditions and a determination result in the leaf node connected by the arrows bolder than the other arrows serve as information indicating determination steps taken by the decision tree 173 and are information about the determination steps. More specifically, in FIG. 3, the part reading “the branching condition of the root node ‘Tumor?’ (Yes)→the branching condition of the node ‘Grade=4?’ (No)→the branching condition of the node ‘IDH mutant?’ (Yes)→the branching condition of the node ‘1p19q Co-deletion?’ (No)→the determination result in the leaf node ‘LGG IDH mutant non co-deletion Final Grade WHO 2’” is displayed with an emphasis, serves as the information indicating the determination steps taken by the decision tree 173, and is the information about the determination steps. Further, the determination result in the leaf node “LGG IDH mutant non co-deletion Final Grade WHO 2” serves as information indicating a determination result and is information about the determination result.

Next, an example of a method for generating the information about the determination steps will be explained. For example, the visualizing function 155c is configured to specify the determination steps of the decision tree 173, by determining whether each of the branching conditions in the decision tree 173 is defined as “Yes” or “No”, by using the tissue information, the gene information, and the molecule information output from the trained model 172. After that, the visualizing function 155c is configured to generate information indicating the specified determination steps.

In the manner described above, the visualizing function 155c and the display 154 are configured to visualize the determination steps that are interpretable by the user such as a medical doctor. In other words, the visualizing function 155c and the display 154 are configured to output (display) the determination steps interpretable by the user such as the medical doctor, together with the determination result. Even more specifically, the visualizing function 155c and the display 154 are configured to output the details 181 of the decision tree 173 indicating inference steps starting from the root node (the branching condition “Tumor?”) and reaching the leaf node indicating the determination result (“LGG IDH mutant non co-deletion Final Grade WHO 2”). Consequently, the medical data processing apparatus 150 according to the first embodiment is able to present the user such as the medical doctor who determines a disease with the information that assists the determination process, in the form that is interpretable by the user. The visualizing function 155c and the display 154 are an example of an output unit, for instance.

Further, as illustrated in FIG. 4, the visualizing function 155c is configured to cause the display 154 to display, together with the details 181 of the decision tree 173, the medical data 171 (171a, 171b, and 171c) input to the trained model 172 as well as a table 182 indicating details of the tissue information, the gene information, and the molecule information. In this situation, “tumor is present” in the tissue information in FIG. 4 corresponds to “1” indicated by the tumor presence information of the tissue information output from the trained model 172. Further, “mutation is present” in the gene information in FIG. 4 corresponds to “1” indicated by the gene information output from the trained model 172. In addition, “deletion is absent” in the molecule information in FIG. 4 corresponds to “0” indicated by the molecule information output from the trained model 172. On the contrary, when the tumor presence information of the tissue information output from the trained model 172 indicates “0”, “tumor is absent” would be written in the table 182. Also, when the gene information output from the trained model 172 indicates “0”, “mutation is absent” would be written in the table 182. In addition, when the molecule information output from the trained model 172 indicates “1”, “deletion is present” would be written in the table 182. In other words, the table 182 is a table indicating the numerical values used when the determining function 155b performs the medical determination processes. In the manner described herein, the visualizing function 155c and the display 154 are configured to visualize, in addition to the determination result and the determination steps, the medical data 171 input to the trained model 172, as well as the intermediate determination items of the tissue information, the gene information, and the molecule information.

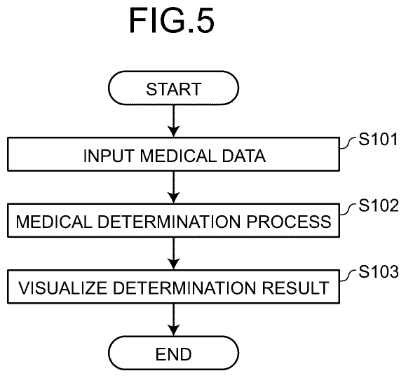

FIG. 5 is a flowchart illustrating an example of a flow in processes performed by the medical data processing apparatus 150 according to the first embodiment.

As illustrated in FIG. 5, the receiving function 155a obtains the medical data from either the medical image diagnosis apparatus group 110 or the medical data storage apparatus 120 via the network 160 and receives the medical data as an input to the trained model 172 (step S101).

After that, the determining function 155b performs the medical determination process by applying the medical determination model including the trained model 172 and the decision tree 173 to the medical data 171 (step S102).

Subsequently, the visualizing function 155c and the display 154 visualize the determination steps that are interpretable by the user such as a medical doctor, together with the determination result (step S103).

The medical data processing apparatus 150 and the medical data processing system 100 according to the first embodiment have thus been explained. According to the first embodiment, it is possible to present the user such as a medical doctor who determines a disease, with the information that assists the determination process, in the form that is interpretable by the user.

### Modification Example of First Embodiment

Next, the medical data processing apparatus 150 according to a modification example of the first embodiment will be explained. In the table 182, for example, there may be a situation where a plurality of numerical values used by the determining function 155b at the time of performing the medical determination process may be inconsistent with each other. For example, the table 182 may indicate “mutation is present” and “deletion is present”, while also indicating “tumor is absent”. In other words, although indicating that no tumor is present, the table 182 may also indicate that the gene that codes IDH in the glioma cell has a mutation and/or that the short arm of chromosome 1 and the long arm of chromosome 19 are both deleted from the glioma cell.

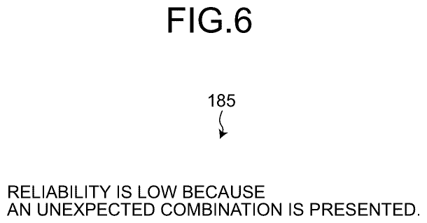

FIG. 6 is a drawing for explaining an example of processes performed by the medical data processing apparatus 150 according to the modification example of the first embodiment. In the situation described above, because the plurality of pieces of information in the table 182 form an unexpected combination, the visualizing function 155c included in the medical data processing apparatus 150 according to the modification example of the first embodiment is configured to cause the display 154 to display, as illustrated in FIG. 6, a message 186 reading “Reliability is low because an unexpected combination is presented” indicating that reliability of the plurality of pieces of information (the numerical values) and reliability of the medical determination result are low. In other words, the visualizing function 155c and the display 154 are configured to output the message 186 indicating that the reliability of the plurality of pieces of information and the reliability of the medical determination result are low, i.e., that the medical determination result is unreliable.

With this arrangement, the user is able to understand that the presented medical determination result is unreliable. Consequently, the modification example of the first embodiment is able to prevent the user from recognizing the wrong medical determination result as a correct medical determination result.

### Second Embodiment

Next, a medical data processing apparatus and a medical data processing system according to a second embodiment will be explained. In the first embodiment, as illustrated in FIG. 4, the table 182 is displayed in addition to the details 181 of the decision tree 173. In that situation, because a large amount of information is displayed at once, the user may find it difficult to easily understand the displayed information. To cope with this situation, the medical data processing apparatus and the medical data processing system according to the second embodiment are configured to enable the user to easily understand the displayed information. In the following description of the second embodiment, some of the elements that are the same as those in the first embodiment will be referred to by using the same reference characters, and the explanations thereof may be omitted. Also, in the description of the second embodiment, differences from the first embodiment will primarily be explained.

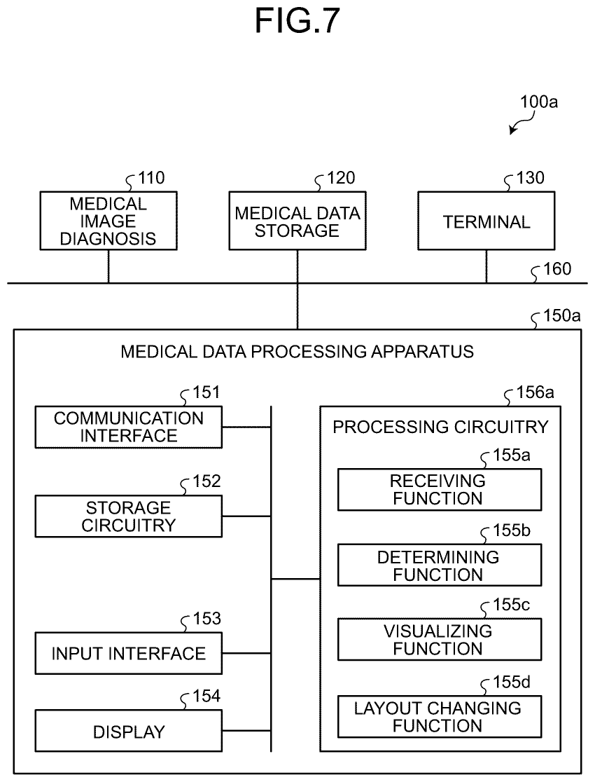

FIG. 7 is a diagram illustrating an exemplary configuration of a medical data processing system 100a and a medical data processing apparatus 150a according to the second embodiment. The medical data processing system 100a according to the second embodiment is different from the medical data processing system 100 according to the first embodiment for including the medical data processing apparatus 150a in place of the medical data processing apparatus 150. The medical data processing apparatus 150a according to the second embodiment is different from the medical data processing apparatus 150 according to the first embodiment for including processing circuitry 156a in place of the processing circuitry 155. The processing circuitry 156a according to the second embodiment is different from the processing circuitry 155 according to the first embodiment for including a layout changing function 155d.

In the second embodiment, the receiving function 155a is configured to receive (transmission of) a layout change instruction input by the user via the input interface 153. In this situation, the layout change instruction is an instruction to change a layout of the display content displayed on the display 154. When the receiving function 155a has received the layout change instruction, the layout changing function 155d is configured to control the visualizing function 155c so as to change the layout of the display content displayed on the display 154. Consequently, when the receiving function 155a has received the layout change instruction, the visualizing function 155c is configured to perform various types of processes described below, under control of the layout changing function 155d.

In the second embodiment, the visualizing function 155c is configured so as not to allow the display 154 to display the details 181 of the decision tree 173 and the table 182 at the same time. More specifically, while the display 154 is displaying the details 181 of the decision tree 173, the visualizing function 155c is configured so as not to allow the display 154 to display the table 182. Conversely, while the display 154 is displaying the table 182, the visualizing function 155c is configured so as not to allow the display 154 to display the details 181 of the decision tree 173.

Even more specifically, when the receiving function 155a has received a layout change instruction while the display 154 is displaying the details 181 of the decision tree 173, the visualizing function 155c is configured to cause the display 154 to display the table 182. As another example, when the receiving function 155a has received a layout change instruction while the display 154 is displaying the table 182, the visualizing function 155c is configured to cause the display 154 to display the details 181 of the decision tree 173. In other words, when the receiving function 155a has received the layout change instruction, the display 154 and the visualizing function 155c are configured to switch the display content so as to display one selected from between the details 181 of the decision tree 173 and the table 182.

Consequently, according to the second embodiment, the details 181 of the decision tree 173 and the table 182 are not displayed at the same time, but are displayed in the mutually-different time periods. It is therefore possible to enable the user to easily understand the details 181 of the decision tree 173 and the table 182.

Further, in the second embodiment, the visualizing function 155c may be configured so as not to allow the display 154 to display the details 181 of the decision tree 173 and the medical data 171 at the same time. More specifically, while the display 154 is displaying the details 181 of the decision tree 173, the visualizing function 155c is configured so as not to allow the display 154 to display the medical data 171. Conversely, while the display 154 is displaying the medical data 171, the visualizing function 155c is configured so as not to allow the display 154 to display the details 181 of the decision tree 173.

Even more specifically, when the receiving function 155a has received a layout change instruction while the display 154 is displaying the details 181 of the decision tree 173, the visualizing function 155c is configured to cause the display 154 to display the medical data 171. As another example, when the receiving function 155a has received a layout change instruction while the display 154 is displaying the medical data 171, the visualizing function 155c is configured to cause the display 154 to display the details 181 of the decision tree 173. In other words, when the receiving function 155a has received the layout change instruction, the display 154 and the visualizing function 155c are configured to switch the display content so as to display one selected from between the details 181 of the decision tree 173 and the medical data 171.

Consequently, the details 181 of the decision tree 173 and the medical data 171 are not displayed at the same time, but are displayed in the mutually-different time periods. It is therefore possible to enable the user to easily understand the details 181 of the decision tree 173 and the medical data 171.

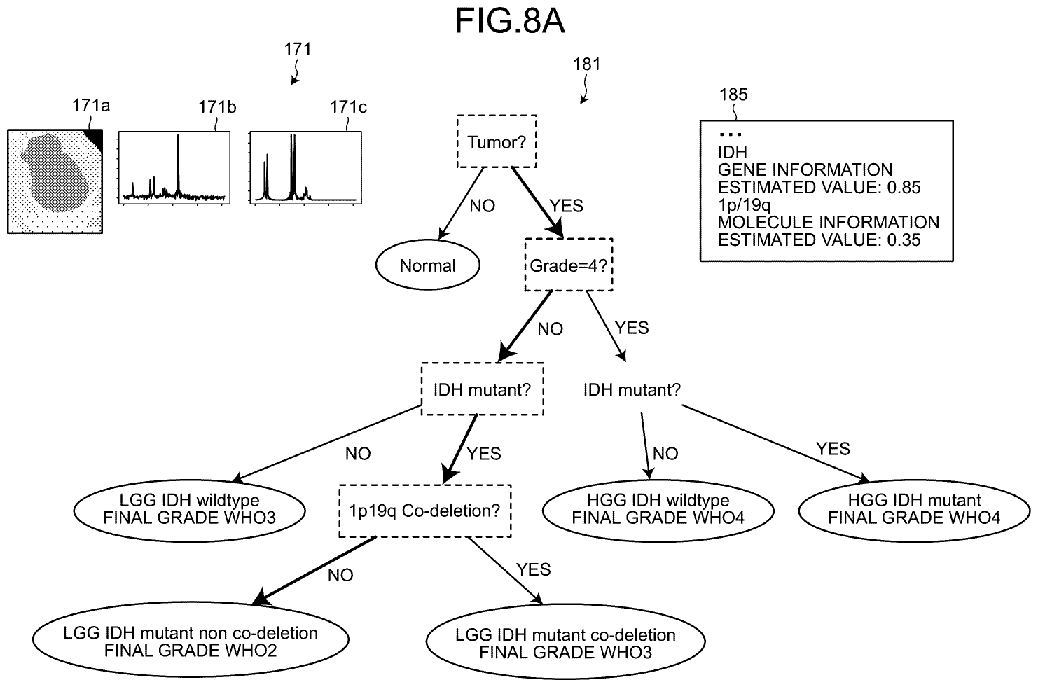

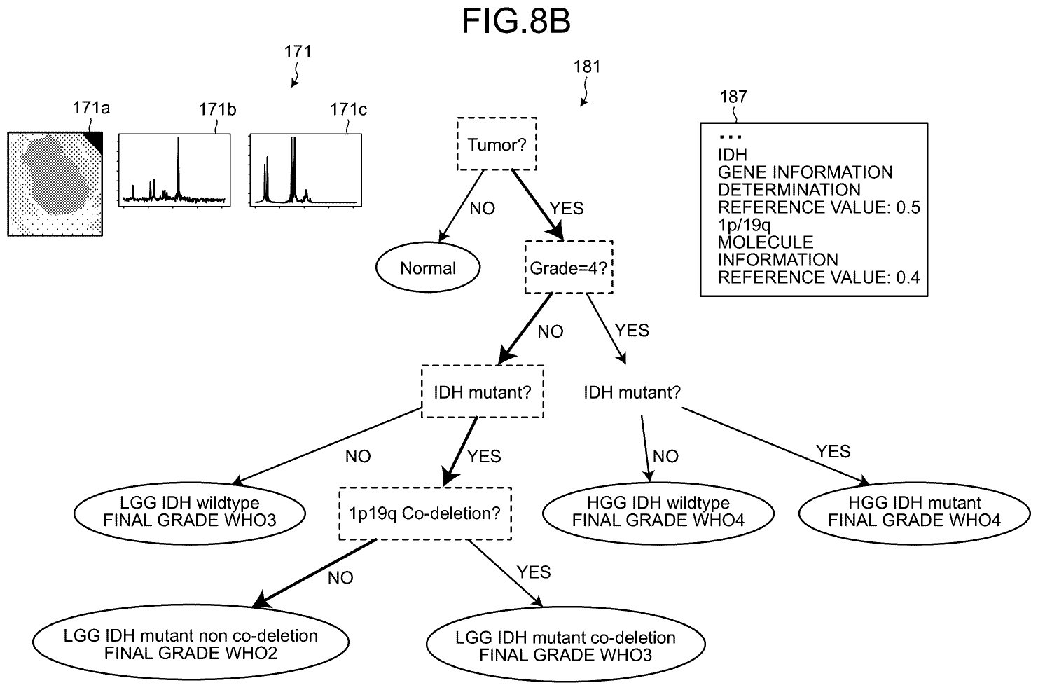

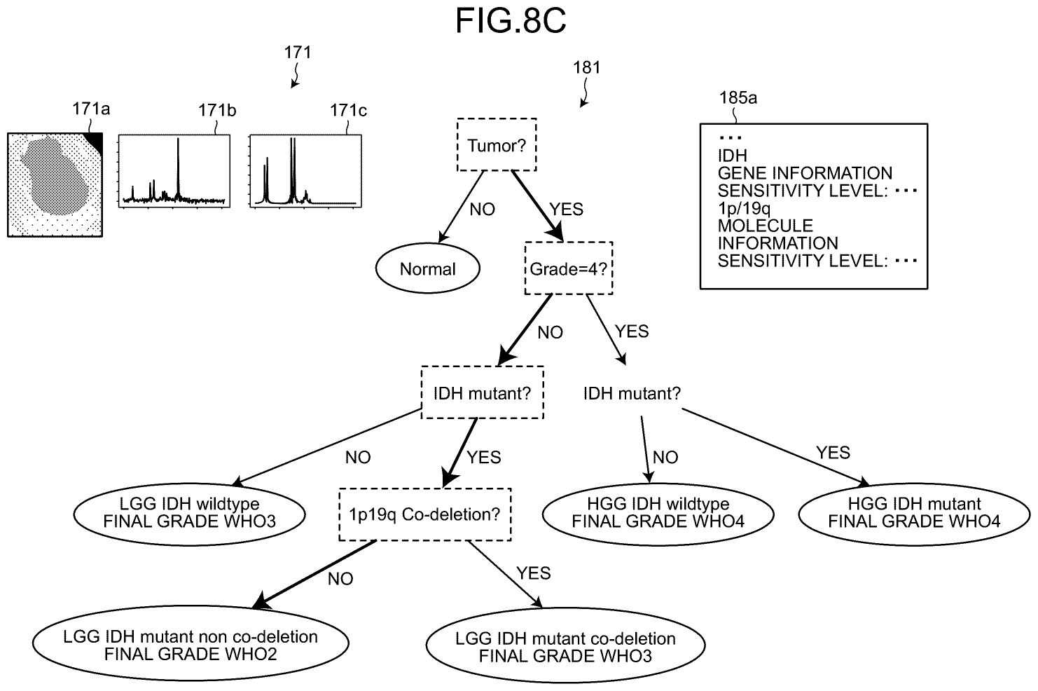

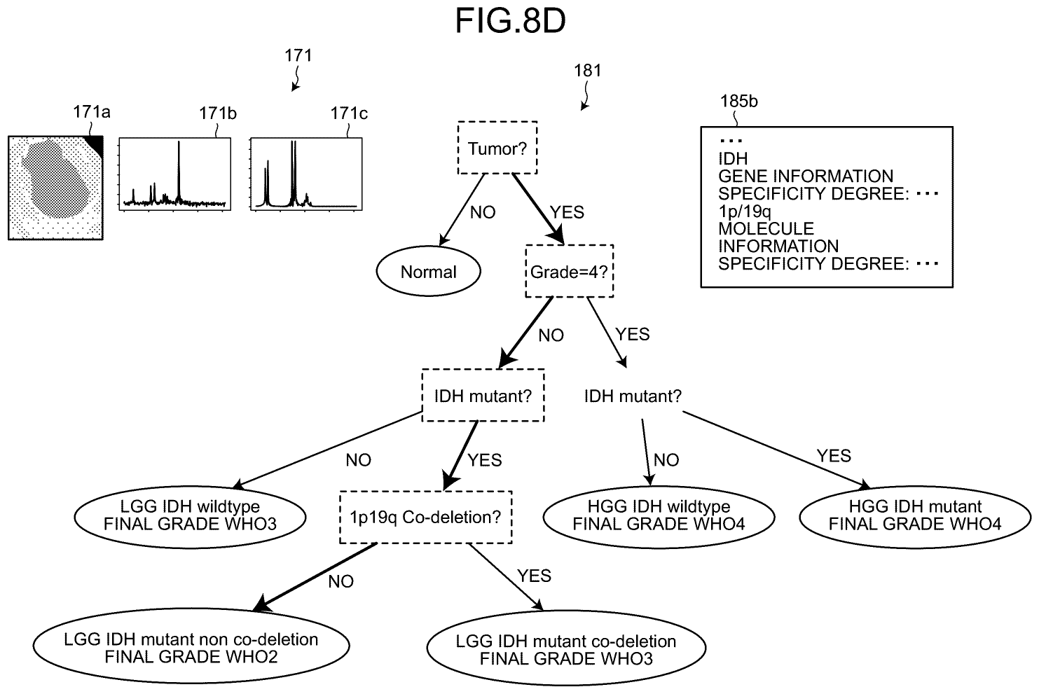

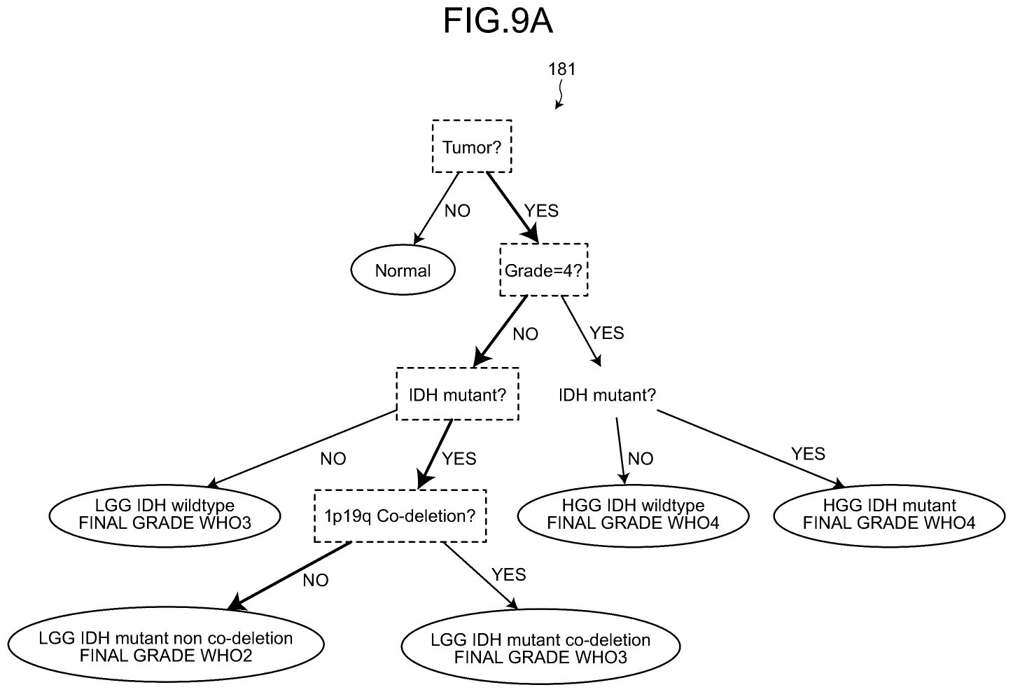

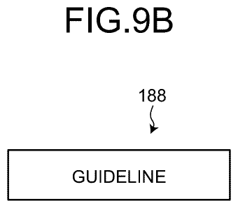

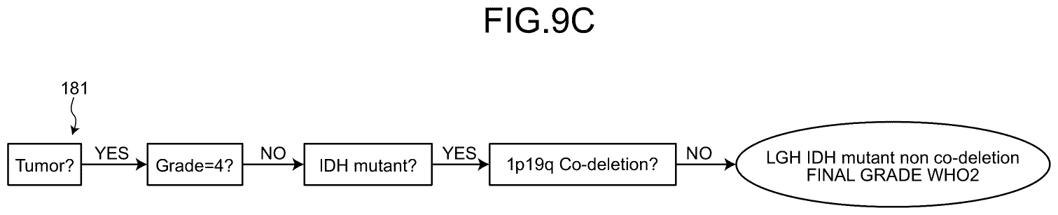

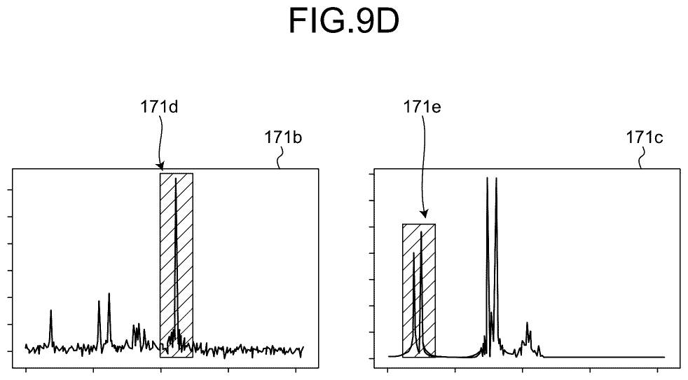

Next, other examples of the switching of the display content in the second embodiment will be explained. FIGS. 8A, 8B, 8C, 8D, 9A, 9B, 9C, and 9D are drawings illustrating examples of various types of display content displayed on the display 154 according to the second embodiment.

For example, as illustrated in FIG. 8A, the visualizing function 155c is configured to cause the display 154 to display the medical data 171, the details 181 of the decision tree 173, and a table 185. In the table 185, the abovementioned probability (the reliability indicating the likelihood of each intermediate determination item) is written with respect to each of the intermediate determination items (the tissue information, the gene information, and the molecule information).

When the receiving function 155a has received a layout change instruction while the display 154 is displaying the display content illustrated in FIG. 8A, the visualizing function 155c is configured to cause the display 154 to display the display content illustrated in FIG. 8B. For example, as illustrated in FIG. 8B, the visualizing function 155c is configured to cause the display 154 to display the medical data 171, the details 181 of the decision tree 173, and a table 187. In the table 187, the determination reference value described above to be compared with the probability is written with respect to each of the intermediate determination items.

Conversely, when the receiving function 155a has received a layout change instruction while the display 154 is displaying the display content illustrated in FIG. 8B, the visualizing function 155c is configured to cause the display 154 to display the display content illustrated in FIG. 8A.

In other words, when the receiving function 155a has received the layout change instruction, the display 154 and the visualizing function 155c are configured to switch the display content so as to display one selected from between the probabilities indicating the likelihood values of the intermediate determination items and the determination reference values.

Consequently, according to the second embodiment, the probabilities indicating the likelihood values of the intermediate determination items and the determination reference values are not displayed at the same time, but are displayed in the mutually-different time periods. It is therefore possible to enable the user to easily understand the probabilities indicating the likelihood values of the intermediate determination items and the determination reference values.

Alternatively, in place of the display content illustrated in FIG. 8A, the visualizing function 155c may be configured to cause the display 154 to display one selected from between: the display content illustrated in FIG. 8C and the display content illustrated in FIG. 8D. For example, as illustrated in FIG. 8C, the visualizing function 155c may be configured to cause the display 154 to display the medical data 171, the details 181 of the decision tree 173, and a table 185a. In the table 185a, a sensitivity level (a true positive rate) is written with respect to each of the intermediate determination items. In this situation, the visualizing function 155c is configured to obtain, from the abovementioned training apparatus, the sensitivity level of each of the intermediate determination items that was calculated at the time of training the trained model 172 and to further generate the table 185a by using the obtained sensitivity levels.

In another example, as illustrated in FIG. 8D, the visualizing function 155c may be configured to cause the display 154 to display the medical data 171, the details 181 of the decision tree 173, and a table 185b. In the table 185b, a specificity degree (a true negative rate) is written with respect to each of the intermediate determination items. In this situation, the visualizing function 155c is configured to obtain, from the training apparatus, the specificity degree of each of the intermediate determination items that was calculated at the time of training the trained model 172 and to further generate the table 185b by using the obtained specificity degrees.

By presenting the user with the evaluation index such as the sensitivity levels or the specificity degrees when the trained model 172 is performing the inference process, it is possible to present the user with the reliability information of the trained model 172. As a result, the user is able to understand capabilities of the trained model 172.

As yet another example, as illustrated in FIG. 9A, the visualizing function 155c may be configured to cause the display 154 to display the details 181 of the decision tree 173. Further, when the receiving function 155a has received a layout change instruction while the display 154 is displaying the display content illustrated in FIG. 9A, the visualizing function 155c is configured to cause the display 154 to display the display content illustrated in FIG. 9B. For example, as illustrated in FIG. 9B, the visualizing function 155c is configured to cause the display 154 to display details of a guideline 188. The guideline 188 is the abovementioned medical guideline that was used at the time of generating the decision tree 173.

Further, when the receiving function 155a has received a layout change instruction while the display 154 is displaying the display content illustrated in FIG. 9B, the visualizing function 155c is configured to cause the display 154 to display the display content illustrated in FIG. 9A.

In other words, when the receiving function 155a has received the layout change instruction, the display 154 and the visualizing function 155c are configured to switch the display content so as to display one selected from between the details 181 of the decision tree 173 and the details of the guideline 188.

Consequently, according to the second embodiment, the details 181 of the decision tree 173 and the details of the guideline 188 are not displayed at the same time, but are displayed in the mutually-different time periods. It is therefore possible to enable the user to easily understand the details 181 of the decision tree 173 and the details of the guideline 188.

As yet another example, the visualizing function 155c may be configured to switch between versions of the guideline 188 of which the details are to be displayed. For example, the visualizing function 155c may include a selecting function. Further, when the selecting function has received, via the input interface 153, an instruction to switch between the versions of the guideline to comply with, while the display 154 is displaying the details of the guideline 188, the display 154 may be caused to display the details of the guideline in the version to which the switching occurred. For example, the guideline is updated once every number of years. Thus, the selecting function is configured to switch between the versions of the guideline of which the details are to be displayed, so as to change, for example, from a version “WHO 2016” to the most up-to-date version “WHO 2021”.

As yet another example, when the receiving function 155a has received a layout change instruction while the display 154 is displaying the display content illustrated in FIG. 9A, the visualizing function 155c is configured to cause the display 154 to display the display content illustrated in FIG. 9C. For example, as illustrated in FIG. 9C, the visualizing function 155c is configured to cause the display 154 to display, out of the decision tree 173, only an inference step 181a starting from a root node (the branching condition “Tumor?”) and reaching a leaf node indicating a determination result (“LGG IDH mutant non co-deletion Final Grade WHO 2”).

Further, when the receiving function 155a has received a layout change instruction while the display 154 is displaying the display content illustrated in FIG. 9C, the visualizing function 155c is configured to cause the display 154 to display the display content illustrated in FIG. 9A.

In other words, when the receiving function 155a has received the layout change instruction, the display 154 and the visualizing function 155c are configured to switch the display content so as to display one selected from between the details 181 of the decision tree 173 and the inference step 181a.

Consequently, according to the second embodiment, the details 181 of the decision tree 173 and the details of the inference step 181a are not displayed at the same time, but are display in the mutually-different time periods. It is therefore possible to enable the user to easily understand the details 181 of the decision tree 173 and the details of the inference step 181a.

In yet another example, when the receiving function 155a has received a layout change instruction while the display 154 is displaying the display content illustrated in FIG. 9A, the visualizing function 155c may be configured to cause the display 154 to display the display content illustrated in FIG. 9D. For example, as illustrated in FIG. 9D, the visualizing function 155c may be configured to cause the display 154 to display only data in which judgment basis information 171d and 171e indicating bases used for judging the intermediate determination items are superimposed on the medical data 171b and 171c. For example, the visualizing function 155c is able to generate the bases used for judging the intermediate determination items, by implementing an existing method such as a Grad-CAM method.

Further, in the example in FIG. 9D, in the medical data 171b and 171c, the parts serving as the bases are indicated with hatching; however, the parts serving as the bases may be indicated with colors. Alternatively, the parts serving as the bases may be indicated with arrows.

As yet another example, when the receiving function 155a has received a layout change instruction while the display 154 is displaying the display content illustrated in FIG. 9D, the visualizing function 155c may be configured to cause the display 154 to display the display content illustrated in FIG. 9A.

In other words, when the receiving function 155a has received the layout change instruction, the display 154 and the visualizing function 155c may be configured to switch between the display content so as to display one selected from between the details 181 of the decision tree 173 and the data in which the judgment basis information 171d and 171e are superimposed on the medical data 171b and 171c.

Consequently, according to the second embodiment, the details 181 of the decision tree 173 and the data in which the judgment basis information 171d and 171e are superimposed on the medical data 171b and 171c are not displayed at the same time, but are displayed in the mutually-different time periods. It is therefore possible to enable the user to easily understand the details 181 of the decision tree 173 and the data in which the judgment basis information 171d and 171e are superimposed on the medical data 171b and 171c.

The term “processor” used in the explanations of the above embodiments denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). In this situation, instead of having the programs saved in the storage circuitry 152, it is also acceptable to directly incorporate the programs in the circuitry of one or more processors. In that situation, the one or more processors are configured to realize the functions, by reading and executing the programs incorporated in the circuitry thereof. Further, the processors of the embodiments do not each necessarily have to be structured as a single piece of circuitry. It is also acceptable to structure one processor by combining together a plurality of pieces of independent circuitry so as to realize the functions thereof.

Further, the programs executed by the one or more processors are provided as being incorporated, in advance, in a Read Only Memory (ROM), storage circuitry, or the like. In that situation, the programs may be provided as being recorded in a non-transitory computer-readable storage medium such as a Compact Disk Read-Only Memory (CD-ROM), a Flexible Disk (FD), a Compact Disk Recordable (CD-R), a Digital Versatile Disk (DVD), or the like, in a file in a format that is installable or executable by those apparatuses. Further, the programs may be stored in a computer connected to a network such as the Internet so as to be provided or distributed as being downloaded via the network. For example, the programs are structured with modules including the processing functions described above. In the actual hardware, as a result of a CPU reading and executing the programs from a storage medium such as a ROM, the modules are loaded into a main storage apparatus and generated in the main storage apparatus.

According to at least one aspect of the embodiments described above, it is possible to present the user such as a medical doctor who determines a disease with the information that assists the determination process, in a form that is interpretable by the user.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical data processing apparatus comprising processing circuitry configured:
to receive medical data as an input;
to perform a medical determination process on a basis of the medical data; and
to output information about a determination step together with a determination result of the medical determination process.

2. The medical data processing apparatus according to claim 1, wherein
a memory has stored therein a medical determination model that is for performing the medical determination process and includes an interpretable model configured to make it possible to interpret the determination step, and
the processing circuitry is configured to perform the medical determination process by applying the medical determination model to the medical data.

3. The medical data processing apparatus according to claim 1, wherein
the medical data is Magnetic Resonance (MR) data, and
the processing circuitry is configured:
to perform the medical determination process including medical classification, on a basis of the MR data; and
to output the information about the determination step together with the determination result including the medical classification.

4. The medical data processing apparatus according to claim 1, wherein
the processing circuitry is configured:
to display the information about the determination step together with the determination result;
to receive a layout change instruction to change a layout of display content being displayed; and
to change the layout of the display content being displayed, upon receipt of the layout change instruction.

5. The medical data processing apparatus according to claim 4, wherein, upon receipt of the layout change instruction, the processing circuitry is configured to switch the display content so as to display, as the information about the determination step, one selected from between: a decision tree that is an interpretable model configured to make it possible to interpret the determination step and that indicates an inference step starting from a root node and reaching a leaf node indicating the determination result; and a table indicating a numerical value used at a time of performing the medical determination process.

6. The medical data processing apparatus according to claim 5, wherein, when two or more of the numerical values in the table are inconsistent with each other, the processing circuitry is configured to output a message indicating that the determination result is unreliable.

7. The medical data processing apparatus according to claim 4, wherein, upon receipt of the layout change instruction, the processing circuitry is configured to switch the display content so as to display, as the information about the determination step, one selected from between: reliability indicating a likelihood of an intermediate determination item that was calculated by a medical determination model performing the medical determination process at a time of obtaining the determination result; and a determination reference value that is to be compared with the reliability and was used by the medical determination model at the time of obtaining the determination result.

8. The medical data processing apparatus according to claim 4, wherein, upon receipt of the layout change instruction, the processing circuitry is configured to switch the display content so as to display, as the information about the determination step, one selected from between: a decision tree that is an interpretable model configured to make it possible to interpret the determination step and that indicates an inference step starting from a root node and reaching a leaf node indicating the determination result; and
a medical guideline that was used at a time of generating the decision tree.

9. The medical data processing apparatus according to claim 4, wherein, upon receipt of the layout change instruction, the processing circuitry is configured to switch the display content so as to display, as the information about the determination step, one selected from between: a decision tree that is an interpretable model configured to make it possible to interpret the determination step and that indicates an inference step starting from a root node and reaching a leaf node indicating the determination result; and
only the inference step, out of the decision tree, starting from the root node and reaching the leaf node indicating the determination result.

10. The medical data processing apparatus according to claim 4, wherein, upon receipt of the layout change instruction, the processing circuitry is configured to switch the display content so as to display, as the information about the determination step, one selected from between: a decision tree that is an interpretable model configured to make it possible to interpret the determination step and that indicates an inference step starting from a root node and reaching a leaf node indicating the determination result; and only the medical data.

11. The medical data processing apparatus according to claim 4, wherein, upon receipt of the layout change instruction, the processing circuitry is configured to switch the display content so as to display, as the information about the determination step, one selected from between: a decision tree that is an interpretable model configured to make it possible to interpret the determination step and that indicates an inference step starting from a root node and reaching a leaf node indicating the determination result; and only data in which judgment basis information is superimposed on the medical data.

12. The medical data processing apparatus according to claim 8, wherein the processing circuitry further comprises a selecting function configured to switch between versions of the medical guideline to comply with.

13. A method comprising:
receiving medical data as an input;
performing a medical determination process on a basis of the medical data; and
outputting information about a determination step together with a determination result of the medical determination process.
